(19) United States
(12) Patent Application Publication
(10) Pub. No.: US 2022/0125998 A1
(43) Pub. Date: Apr. 28, 2022

(54) MEMBRANE

(71) Applicant: LES LABORATOIRES OSTEAL MEDICAL, ROISSY AEROPORT CDG (FR)

(72) Inventors: Daniel BLANQUAERT, PARIS (FR); Amélie LEROUX, ERMONT (FR); Véronique MIGONNEY, EAUBONNE (FR)

(73) Assignee: LES LABORATOIRES OSTEAL MEDICAL, ROISSY AEROPORT CDG (FR)

(21) Appl. No.: 17/432,579

(22) PCT Filed: Feb. 18, 2020
(86) PCT No.: PCT/FR2020/050290

§ 371 Date: Aug. 20, 2021

(30) Foreign Application Priority Data
Feb. 22, 2019 (FR) 1901812

(51) Int. Cl. A61L 31/04; A61L 31/14; A61L 31/10; A61L 15/26; A61L 15/64; C08L 67/04; C09D 125/18; D04H 1/435; D04H 1/45; A61L 15/18

(52) U.S. Cl. CPC A61L 31/04; A61L 31/148; A61L 31/10; A61L 15/26; A61L 15/18; C08L 67/04; C09D 125/18; D04H 1/435; D04H 1/45; A61L 15/64

(57) ABSTRACT

Membrane, used in particular for regenerating periodontal ligament, preventing the formation of adhesions or the regeneration and reconstruction of tissue, including at least one layer of fabric (N1) made of fibres (F) produced from at least one polymer. The fibres (F) are free of sizing, the layer of fabric (N1) has a non-woven arrangement of fibres disposed in a random or aligned manner, and the fibres (F) are coated with PolyNaSS (polystyrene sodium sulfonate).

The present invention relates to a membrane and to a method of manufacturing the same. This membrane comprises at least one layer of fabric made of fibres produced from at least one polymer. This type of membrane can in particular be used for tissue regeneration and reconstruction, including skin tissues and periodontal ligament; for preventing the formation of adhesions or also in dressings to regenerate tissues and to minimise risks of infections.

In the dental field, it is known to implant this type of membrane between the gum and the root of the tooth, with a view to regenerating the periodontal ligament, which may be damaged, for example in the case of periodontal diseases. Regeneration is both tissue and bone. The membrane makes it possible to guide the regeneration.

In the maxillofacial field, membranes can be used to lift the sinuses.

This type of membrane is also used in various surgical specialties in order to avoid tissue adhesions.

In the field of orthopaedic surgery, for example, various surgical solutions have been proposed in order to prevent the formation of adhesions after surgery: drug treatment, advancement (fatty) flaps, use of membranes. Local flaps may be beneficial, but often the result is unpredictable. The membranes are interposed between the damaged tissues and the surrounding tissues so as to prevent fibrinotic adhesions, which are inevitable after any visceral, tendon and/or nerve surgery. In orthopaedic surgery, in particular post-traumatic surgery, the absence of effective products often requires iterative tenolysis and/or neurolysis interventions.

Collagenic membranes, designed to guide the healing process and prevent the formation of adhesions, can be composed of 100% collagen. Due to its animal origin (cattle or pigs in general), any risk of contamination associated with Bovine Spongiform Encephalopathy (BSE) or mad cow disease cannot be excluded.

There are also other synthetic membranes, including:

resorbable membrane composed of hyaluronic acid and carboxymethylcellulose,

resorbable cellulose membrane,

non-absorbable barrier of poly(tetrafluoroethylene) (PTFE),

poly(ethylene glycol) (PEG) absorbable membrane.

There are therefore a large number of different membranes, of animal, plant or synthetic origin, which may or may not be resorbable, in the form of a film or tissue.

As regards tissues, i.e. the fabrics woven with a weft and a warp, or else knitted, their manufacture requires sizing of the yarns or fibres, which consists in impregnating the yarns or fibres, generally at the time of extrusion, with a protective molecule known as a sizing molecule. This sizing molecule, generally an oil of an organic nature, imparts to the yarns or fibres a certain strength and protects them during various subsequent manipulations (weaving, knitting, winding). However, in the case of woven membranes to be implanted in the body, it is necessary to remove the sizing molecule to avoid its “release” in situ responsible for acute or even chronic inflammation: desizing is referred to. This operation, which in itself is expensive, is industrially difficult. In addition, an effective desizing requires the use of organic solvents which can damage the fibres of the membrane.

On the other hand, the fibres of the membrane do not per se impart exceptional anti-adhesion qualities.

The aim of the present invention is to overcome the drawbacks of the prior art by defining a novel membrane that solves the problems associated with desizing and that exhibits very high performance in terms of anti-adhesion.

To do this, the present invention proposes a membrane, used in particular for regenerating the periodontal ligament or for preventing the formation of adhesions, comprising at least one layer of fabric made of fibres produced from at least one polymer, characterised in that:

the fibres are free of sizing,

the layer of fabric has a non-woven arrangement of fibres disposed in a random of aligned manner, and

the fibres are coated with polyNaSS (polystyrene sodium sulfonate).

Thus, the membrane of the invention has three distinct characteristics, namely the absence of sizing product, the non-woven nature of the layer of fabric and a coating, giving it the following properties and advantages:

Fibre purity,

Isotropy in the case of randomly arranged fibres,

Variable thickness,

Uniformity in thickness,

Anti-bacterial adhesion of PolyNaSS,

Tissue regeneration and reconstruction of PolyNaSS.

According to another advantageous characteristic of the invention, the polymer is a polyester, such as, for example, poly(ε-caprolactone) (PCL) or poly(dioxanone) (PDO). A mixture of polymers to form a copolymer is also possible. A copolymer of poly(ε-caprolactone) (PCL) and poly(dioxanone) (PDO) is particularly advantageous.

Indeed, PCL has non-inflammatory properties (because it does not release acid by degrading), it is a viscoelastic polymer, with slow biodegradation, which is favourable to tissue reconstruction. PDO also has non-inflammatory properties, rapid biodegradation, and is easily removed from the body by through urine. The PCL-PDO copolymer makes it possible to modulate the parameters of the two polymers taken independently, in particular to modulate the rate of biodegradation.

The polymer may also be a polyester, for example polyethylene glycol terephthalate (PET), or alternatively a polyolefin, for example polypropylene (PP). PET has good mechanical strength. It is temperature and UV stable. The grafting of PolyNaSS onto this material is already well-controlled. PP is economical, highly resistant to fatigue and bending, and easily sterilizable.

A biopolymer, for example cellulose, could also be used.

According to another aspect of the invention, the PolyNaSS is grafted onto the fibres. In a variant, the PolyNaSS may be applied to the fibres by dipping, spraying, or any other known method.

According to an embodiment, the layer of fabric has a thickness and a fibre density, the fibre density then being able to vary through the thickness with a gradual or stepwise gradient. Conversely, the fibre density may be constant across the thickness in another embodiment. This capacity to vary fibre density and thickness is not possible with a woven fabric.

According to an embodiment, the membrane may comprise a layer of fabric of the invention disposed on a substrate. In a variant, the membrane may comprise two layers of fabric of the invention disposed on either side of a substrate. The two layers of fabric may be identical or different, both in thickness and in density, and also in the nature of a polymer.

Advantageously, the substrate is less porous than the layer of fabric. The substrate thus constitutes a barrier that prevents or hinders the tissues from growing through the substrate, thus avoiding adhesions.

The substrate may be chosen from a film of poly(ε-caprolactone) (PCL), a film of poly(dioxanone) (PDO), a film of polyethylene glycol terephthalate (PET), a film of poly(propylene) (PP) or also a non-woven fabric web, for example made of poly(caprolactone) (PCL) and/or poly(dioxanone) (PDO), having a fibre density greater than that of the layer of fabric. A cellulose film or a web (woven or non-woven) may also be used as a substrate.

The present invention also defines a multilayer structure comprising:

a ceramic substrate, advantageously tricalcium phosphate, and preferably tricalcium β-phosphate (βTCP),

a membrane such as defined above, advantageously made of poly(ε-caprolactone) (PCL), and

optionally, a cohesion interface, disposed between the ceramic substrate and the membrane.

This multilayer structure may be used for the treatment of osteoarthritis, but also for cartilage reconstruction. The present invention also defines a dressing intended to be applied to a skin wound, the dressing comprising a substrate and at least one membrane such as defined above.

The present invention also defines a method of manufacturing a membrane, used in particular for the regeneration of the periodontal ligament, comprising at least one layer of fabric formed of fibres made from at least one polymer, the method comprising the following steps:

a) obtaining polymer fibres free of sizing,
b) depositing said polymer fibres in a random or aligned manner, so as to form a non-woven layer of fabric,
c) coating the fibres of the non-woven layer of fabric, for example by grafting or dipping, with PolyNaSS (polystyrene sodium sulfonate).

The polymer may be poly(ε-caprolactone) (PCL), poly(dioxanone) (PDO), a copolymer of poly(ε-caprolactone) (PCL) and poly(dioxanone) (PDO), polyethylene glycol terephthalate (PET) or polypropylene (PP).

A non-woven layer of fabric may be deposited on one or both sides of a substrate chosen from a film of poly(ε-caprolactone) (PCL), a film of poly(dioxanone) (PDO), a film of polyethylene glycol terephthalate (PET), a film of polypropylene (PP) or a non-woven layer of fabric, for example made of poly(ε-caprolactone) (PCL) and/or poly(dioxanone) (PDO), having a fibre density greater than that of the layer of fabric.

The scope of the invention resides in combining a non-woven layer that is free of sizing with a bacterial release coating of PolyNaSS. The non-woven layer may be produced of biodegradable polymer or copolymer. It may be associated with a substrate and/or with another non-woven layer. The membrane of the invention may also integrate other elements of components, like a ceramic substrate. Thanks to the invention, it is possible to manufacture periodontal membranes, anti-adhesion membranes, hernia reinforcement meshes, dressings or alternatively multilayer structures used for the treatment of arthrosis.

The invention will now be described in more detail with reference to the accompanying drawings, which show several embodiments of the invention as non-limiting examples.

In the figures:

FIG. 1 is a perspective, schematic view of a non-woven layer of fabric with random fibres having a constant fibre density,

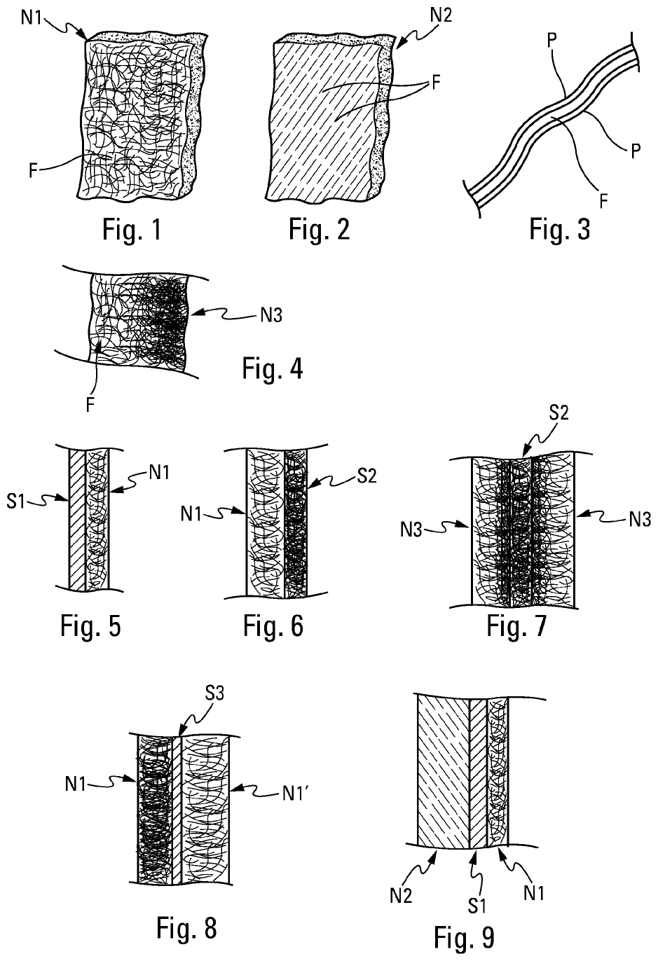

FIG. 2 is a perspective, schematic view of a non-woven layer of fabric with aligned fibres having a constant fibre density,

FIG. 3 is a highly enlarged cross-sectional view of a fibre coated with PolyNaSS,

FIG. 4 is a transversal cross-sectional view through a non-woven layer of fabric with random fibres having an increasing fibre density or which has a progressive gradient,

FIG. 5 is a transversal cross-sectional view through a membrane comprising a substrate in the form of a film and a non-woven layer of fabric with random fibres,

FIG. 6 is a transversal cross-sectional view through a membrane comprising a substrate in the form of a non-woven fabric web with random fibres and a non-woven layer of fabric with random fibres,

FIG. 7 is a transversal cross-sectional view through a membrane comprising a substrate in the form of a non-woven fabric web with random fibres of constant fibre density and two identical non-woven layer of fabrics by symmetry with random fibres of increasing fibre density, and

FIG. 8 is a transversal cross-sectional view through a membrane comprising a substrate in the form of a film and two non-woven layer of fabrics with random fibres of different constant fibre densities,

FIG. 9 is a transversal cross-sectional view through a membrane comprising a thick substrate in the form of a film and two non-woven layer of fabric, one of which with random fibres and another with aligned fibres,

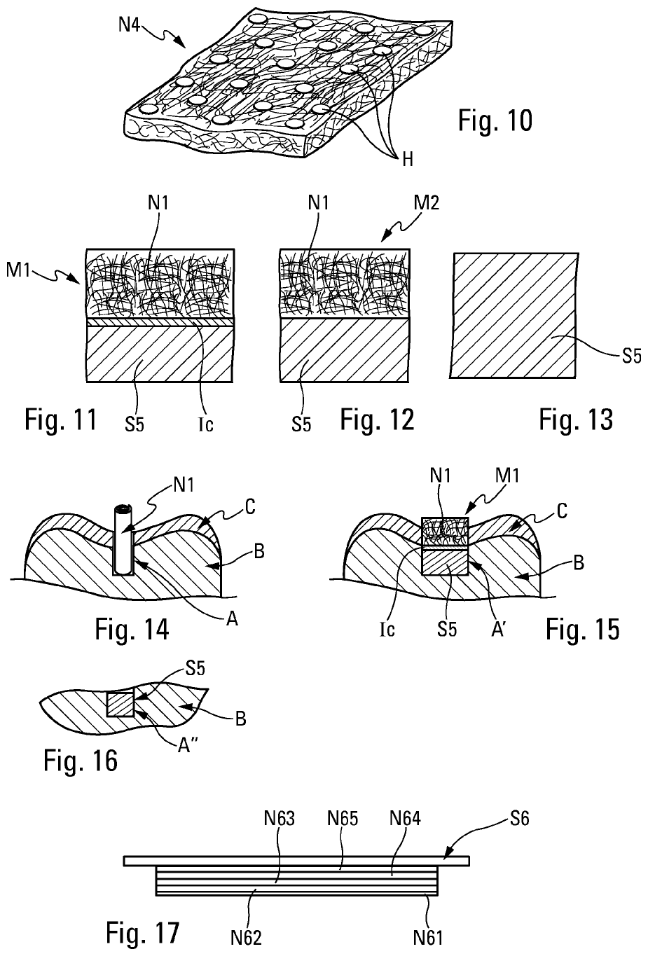

FIG. 10 is a schematic perspective view of a non-woven layer of fabric with random fibres provided with holes,

FIG. 11 is a transversal cross-sectional view through a multilayer structure integrating the membrane of the invention,

FIG. 12 is a view similar to that of FIG. 11 for a multilayer structure variant,

FIG. 13 is a transversal cross-sectional view of a βTCP block coated with PolyNaSS,

FIG. 14 is a schematic, cross-sectional view of a joint treated with a membrane of the invention,

FIG. 15 is a schematic, cross-sectional view of a joint treated with the multilayer structure of FIG. 12,

FIG. 16 is a schematic, cross-sectional view of a bone treated with the TCP block of FIG. 13, and

FIG. 17 is a transversal, cross-sectional view of a dressing integrating several membranes of the invention.

The basic entity of the membrane of the invention is a non-woven layer of fabric made from polymer fibres disposed in a random or aligned manner. By “non-woven”, this means any product manufactured from fibres of which the internal cohesion is ensured by mechanical and/or physical and/or chemical methods and/or by a combination of these various methods, with the exception of weaving and knitting. The fibres may be aligned in a particular direction or at random in the form of a non-repetitive entanglement. The felt may be considered as non-woven.

FIG. 1 shows a piece of non-woven layer of fabric, the fibres F of which are disposed in a random manner, while FIG. 2 shows the fibres F disposed in an aligned manner. All the fibres F may have the same alignment, or, in a variant, they may have different determined alignments, for example from one layer to the other.

According to the invention, the fibres F are free from sizing and are therefore pure. Indeed, it is not necessary to size them, as this would be the case with a woven layer of fabric, given that they are not subjected to significant stresses: they are simply deposited one on top of the other, and possibly compressed.

The fibres F are made from polymer(s) or copolymer(s), or even from biopolymer. For example, it is possible to use a polyester, such as poly(caprolactam) (PCL) or poly(dioxanone) (PDO). A copolymer of poly(ε-caprolactone) (PCL) and poly(dioxanone) (PDO) is a good candidate, because both PCL and PDO are biodegradable, but with different degradation times, PCL degrading quicker than PDO. Their copolymer therefore has a longer and staggered degradation time.

Poly(caprolactone) (PCL) is a biodegradable polyester with a low melting point of about 60° C. and a glass transition temperature of about −60° C. It may also be designated by the term poly(ε-caprolactone).

Other polyester fibres, which may or may not be biodegradable, may also be used, for example polyethylene glycol terephthalate (PET).

It is also possible to use a polyolefin, such as poly(propylene) (PP).

Cellulose can also be used.

According to the invention, the fibres are coated with PolyNaSS (polystyrene sodium sulfonate). For example, PolyNaSS may be grafted onto the fibres. Any grafting method or technique may be used, like for example as that described in document FR3042715, which describes a method of grafting PolyNaSS onto titanium implants, but this method may also be used on membranes of the invention. The grafting method of this document can be adapted to the grafting of fibres and can, in this case, provide the following successive steps:

a) Mounting implants on a support structure,
b) Activating the fibre surface by generating peroxide or hydroperoxide functions by ozonation or by UV irradiation,
c) Putting the membranes (still mounted on their support, or on another support, or on no support) into a gastight polymerisation chamber filled with inert gas, such as argon: another inert gas may also be used,
d) Mounting the membranes (mounted on their support, or on another support, or on no support) onto an elevator installed in the gastight chamber,
e) Actuating the elevator so as to dip the membrane into a polymerisation bath, for example a bath of monomer, such as styrene sodium sulfonate (NaSS), present in the chamber,
f) Subjecting the polymerisation bath to an energy, for example thermal or UV, to polymerize the bioactive monomer from the fibre surface, and to thus obtain a membrane coated with a layer of grafted polymer, for example PolyNaSS,
g) Raising the elevator to extract the coated membranes from the polymerisation bath,
h) Removing the membranes from the elevator,
i) Extracting the membrane from the gastight chamber,
j) Washing the membrane, for example by spraying pure water, to remove any excess non-grafted bioactive polymer therefrom,
k) Possibly drying the grafted membranes.

This succession of steps makes it possible for an industrialised implementation of the grafting method. Most steps are essential, even vital for an industrialised, reproducible, effective, quick and reliable implementation of the method of grafting bioactive polymer.

Instead of grafting, it is also possible to apply PolyNaSS to the fibres of the membrane by using, for example, dipping or spray techniques.

PolyNaSS is known for its anti-bacterial adhesion qualities, resulting in a lower risk of adhesion of bacteria that can cause infections, and for its regeneration, repair and tissue reconstruction qualities.

Thus, the invention proposes a synthetic membrane integrating a non-woven layer of fabric of unsized fibres coated with PolyNaSS. FIG. 3 shows a cross-section of fibre F coated with a layer P of PolyNaSS: there is no sizing layer.

In FIG. 4, shows a non-woven layer of fabric of unsized fibres coated with PolyNaSS disposed in a random manner. However, it can be noted that the fibre density is not constant over the thickness of the layer: on the contrary, it varies according to a progressive continuous gradient from left to right in FIG. 4. The progression of the gradient may be linear or, on the contrary, stepwise. It would be possible to consider a layer with several layers of different densities.

According to a simple embodiment, the membrane of the invention may consist solely of a non-woven layer of fabric of unsized fibres coated with PolyNaSS, like those illustrated in FIGS. 1, 2 and 4. The thickness of the layer of fabric may be from 0.1 to 3 mm, advantageously from 0.1 to 0.8 mm, and preferably from 0.2 to 0.4 mm.

The layer may however be combined with other elements or components. FIGS. 5 to 9 illustrate complex membranes according to the invention. A layer according to the invention may be associated with a substrate and/or with another layer of the invention.

The substrate may be of any nature, structure, thickness, and shape. It may be chosen, for example, from a film of poly(ε-caprolactone) (PCL), a film of poly(dioxanone) (PDO), a film of polyethylene glycol terephthalate (PET), a film of poly(propylene) (PP) or a non-woven fabric web, for example made of poly(ε-caprolactone) (PCL) and/or poly(dioxanone) (PDO), having a fibre density greater than that of the layer of fabric. A cellulose film or web (woven or non-woven) may also be used as a substrate. Advantageously, the substrate is less porous than the layer. Thus, it can act as a barrier. The thickness of the film may be 0.05 to 0.5 mm, advantageously 0.05 to 0.3 mm, and preferably 0.05 to 0.25 mm.

In FIG. 5, the membrane comprises a substrate S1 in the form of a film and a non-woven layer of fabric N1 with random fibres of constant density, which may be identical or similar to that of FIG. 1. The layer is thicker than the substrate. The layer N1 may be simply deposited on the film S1 or, in a variant, the layer N1 may be bonded to the substrate S1, or vice versa.

In FIG. 6, the membrane comprises a substrate S2 in the form of a non-woven fabric web with random fibres and a non-woven layer of fabric N1 with random fibres of constant density, which may be identical or similar to that of FIG. 1. The substrate S2 and the layer N1 therefore have an identical non-woven structure. However, it may be observed in FIG. 6 that the fibre density of the substrate S2 is greater than that of the layer N1. It can also be said that the layer N1 is more porous than the substrate S2.

In FIG. 7, the membrane comprises a substrate S2 in the form of a non-woven fabric web with random fibres of constant fibre density and two non-woven layers of fabric N3 identical by random fibre symmetry, but of increasing fibre density. The layers N3 may be identical or similar to that of FIG. 4. The fibre density of the substrate S2 is greater than or equal to the maximum density of the layers N3.

In FIG. 8, the membrane comprises a substrate S3 in the form of a thin film and two non-woven layers of fabric N1 and N1′ with random fibres of different constant fibre densities. Indeed, it can be seen that the density of the layer N1 is greater than that of the layer N1′.

In FIG. 9, the membrane comprises a substrate in the form of a thick film and two non-woven layers of fabric N1 and N2, one of which has random fibres and the other has aligned fibres. The layer N1 may be identical or similar to the layer in FIG. 1 and the layer N1 may be identical or similar to that of FIG. 2.

In the examples of FIGS. 5 to 9, the total thickness of the multilayer membrane may vary from 0.1 to 3 mm, advantageously from 0.1 to 0.8 mm, and preferably from 0.2 to 0.4 mm.

From these multiple examples, it will be understood that it is possible to produce a multitude of membranes of different designs, ranging from one single layer to an assembly of layer(s) and substrate(s). More than two layers and more than one substrate can be provided, without moving away from the scope of the invention.

The layer of fabric of the invention can be produced from polymer fibres (F) free from sizing, which are deposited without random or aligned weaving or knitting, in order to obtain a non-woven layer of fabric having a constant or progressive thickness and density. Then, PolyNaSS (polystyrene sodium sulfonate) is applied (grafting, dipping, spraying) on the fibres of the non-woven layer of fabric. The layer may be made directly on the substrate so as to adhere mechanically and/or chemically thereto.

FIG. 10 shows a membrane N4, made of a non-woven layer of fabric coated with PolyNaSS, which comprises holes H, advantageously disposed in a regular manner in the form of a network. The membrane is thus perforated. The holes H may have a round, square, rectangular, hexagonal cross-section, etc. The holes may represent up to 90% of the surface of the membrane, such that it then resembles a mesh or a net. Holes H may be made by punching or laser cutting, for example. The membrane may be flat or calendered. Such a membrane may be used as a hernia reinforcement mesh. The risk of infection is decreased through the presence of PolyNaSS and the tissue reconstruction is done through the holes H.

FIG. 11 highly schematically shows a complex multilayer structure M1 comprising:

a ceramic substrate S5, which may comprise or consist of tricalcium phosphate, and more specifically tricalcium phosphate β (βTCP),

a cohesion interface Ic, which may be made by impregnating a polymer, such as poly(ε-caprolactone) (PCL), and

a membrane, made of a non-woven layer of fabric coated with PolyNaSS, such as the membrane N1 of FIG. 1.

Tricalcium phosphates are whitish solids: they make up the mineral part of bones and teeth. β-tricalcium phosphate (βTCP) is a macroporous ceramic having a pore size ranging from 250 to 400 μm. Sterilisation is provided by gamma irradiation. This ceramic is perfectly biocompatible, bioactive and of synthetic origin, which avoids any problem of immune intolerance and eliminates any risk of viral transmission. Moreover, it is osteoconductive and resorbable.

The cohesion interface Ic may be made by applying a liquid polymer to one face of the substrate S5. The abovementioned polymers can be used, with a preference for poly(ε-caprolactone) (PCL). The function of this cohesion interface Ic is to provide the mechanical connection between the substrate S5 and the membrane of the invention.

The membrane of the invention may be any one of FIG. 1, 2, 3, 6 or 7, or also another single-layer or multilayer non-woven membrane coated with PolyNaSS. Only the membrane of the invention may be coated with PolyNaSS. In a variant, the entire complex multilayer structure may be coated with PolyNaSS.

FIG. 12 highly schematically shows a variant of the multilayer structure M2, less complex than the one in FIG. 11, since it does not comprise a cohesion interface. The membrane of the invention is then directly in contact with the substrate S5. For example, the membrane may be made directly on the substrate S5 by successive deposition of fibres. The PolyNaSS coating, pure or additive, may then be grafted, dipped or sprayed onto the membrane and the substrate: it may optionally contribute to the bond between the membrane and the substrate S5.

FIG. 13 highly schematically shows the substrate S5 coated with PolyNaSS. Preferably, the ceramic substrate S5 is a bone substitute and may comprise tricalcium phosphate, and more specifically, tricalcium β-phosphate (βTCP). PolyNaSS is preferably grafted, but it may also be applied by dipping or spraying in certain cases. Protection could be sought for such a ceramic substrate coated with PolyNaSS.

FIGS. 14 and 15 illustrate treatment methods using the membrane of the invention in different forms. It shows a bone part comprising cancellous bone B and cartilage C which covers the cancellous bone B. In the event of damage to the cartilage C, and possibly to the cancellous bone B, for example due to osteoarthritis or trauma, the invention provides for a bore A, A′ to be made which passes through the cartilage C and possibly through the cancellous bone B, as shown in FIGS. 14 and 15. Without moving away from the scope of the invention, the bore A, A′ could stop before reaching the cancellous bone B, such that the bottom of the bore would be made by cartilage C.

In the case of FIG. 14, a membrane of the invention is inserted into the bore A. The membrane may be any one of FIG. 1, 2, 3, 6 or 7, or also another single-layer or multilayer non-woven membrane coated (grafted, dipped or sprayed) with PolyNaSS. The membrane can be wound on itself to form a small cylinder that is then inserted into the bore A. After full insertion, the membrane expands in the bore by pressing against the inner wall of the bore A. The small cylinder of the membrane can project out of the bore A, as can be seen in FIG. 14, or, alternatively, the small cylinder can be adjusted or cut such that it comes substantially flush with the outer surface of the cartilage C. Instead of the cylinder, it can also be considered to form a membrane that fits into the bore A: the membrane can have the form of a small rod. More simply, it is possible to drive (by jamming) a membrane into the bore A and to cut or not cut what protrudes.

Here, the interest is in preventing infection for the entire device, cartilage reconstruction and ultimately the reduction of osteoarthritis.

In the case of FIG. 15, a small rod made from the complex multilayer structure M1 is inserted in the bore A′. The substrate S5 is disposed in the cancellous bone B, while the membrane N1 is disposed in the cartilage C. The cohesion interface Ic is located about at the interface between the cancellous bone B and the cartilage C.

Here, the interest is in preventing infection for the whole device, cartilage reconstruction, to which is added the improvement of bone reconstruction for the bone part and ultimately the reduction of osteoarthritis.

FIG. 16 shows a bone B wherein a bore A″ has been bored, then filled with an adhesive disc or substrate rod S5 of FIG. 13, namely a ceramic coated with PolyNaSS, and preferably with β-tricalcium phosphate (βTCP) grafted, dipped or sprayed with PolyNaSS.

This technique advantageously replaces the various repair techniques with autologous chondrocytes obtained by cultures (Carticel® technique). All these techniques, including the one described in the patent aim to obtain a healthy cartilage and not a fibrocartilage.

Here, the interest is clearly in preventing infection to which is added the improvement of bone reconstruction.

The membrane of the invention can also be used for the design of dressings for skin wounds, intended to protect various wounds, and more specifically, extended wounds, such as burns, abrasions, varicose ulcers, etc. A dressing according to the invention comprises a substrate S6 and one, but preferably more, membranes of the invention N61 to N65. The substrate S6 may be entirely conventional and may be in the form of a flexible strip of plastic material. The membranes may be identical, but preferably they are different. For example, the outermost membrane 61 may be very thin and more strongly grafted (high grafting rate) than the second membrane 62. The porosity or density of the membrane 61 may also be greater than that of the second membrane 62. The membranes 62 to 65 may be identical or different in the manner of the membrane 61 with respect to the membrane 62. Thus, a sort of millefeuille of membranes with distinct characteristics is obtained. The membranes may be made from a non-woven polymer layer, as defined above. Preferably, the polymer is poly(ε-caprolactone) (PCL).

In practice, after having kept this dressing on a wound for a certain time, it is possible to remove it by leaving one or more membranes in place, which will thus constitute a kind of second skin. It can be said that the membranes are “peelable”, with the outer membrane 61 which forms a thin artificial skin with a high PolyNaSS content. This significantly reduces the risk of infection, while accelerating tissue regeneration.

It must also be noted that the degree of grafting of PolyNaSS varies from one application to the other. For example, it is possible to provide very low grafting rates, close to zero, for cellular anti-adhesion, because ozonisation makes it possible to create hydroperoxides that will be hydrolysed and will generate OH at the surface that will make them hydrophilic and therefore non-adherent for the cells. In practice, a PolyNaSS grafting rate of 0.01 to 0.1 micromoles per g or cm² gives good results for cell anti-adhesion. Conversely, it is possible to provide very high grafting rates, up to 40 micromoles per g or cm² for bacterial anti-adhesion and tissue regeneration. In practice, a PolyNaSS grafting rate of 12 micromoles per g or cm² gives good results for bacterial anti-adhesion.

Thus, thanks to the invention, a synthetic membrane is available that can be used in dental surgery for regenerating the periodontal ligament, but also in various surgical specialties to prevent the formation of adhesions, or also for tissue regeneration and reconstruction (reinforcing mesh for hernias), including skin tissues, for example in the form of dressings to minimise the risks of infections and to accelerate regeneration.

1. A membrane, used in particular for regenerating the periodontal ligament, preventing the formation of adhesions or tissue regeneration and reconstruction, comprising at least one layer of fabric (N1, N1′, N2, N3; N4; N61, N62, N63, N64, N65) made from fibres (F) produced from at least one polymer, wherein:
the fibres (F) are free of sizing,
the layer of fabric (N1, N1′, N2, N3; N4; N61, N62, N63, N64, N65) has a non-woven arrangement of fibres disposed in a random or aligned manner, and
the fibres (F) are coated with PolyNaSS (polystyrene sodium sulfonate).

2. The membrane according to claim 1, wherein the polymer is selected from poly(ε-caprolactone) (PCL), poly(dioxanone) (PDO), a copolymer of poly(ε-caprolactone) (PCL) and poly (dioxanone) (PDO), polyethylene glycol terephthalate (PET), and polypropylene (PP).

3. The membrane according to claim 1, wherein the layer of fabric (N4) comprising holes (H).

4. The membrane according to claim 1, wherein the layer of fabric (N3) has a thickness and a fibre density, the fibre density varying through the thickness with a progressive gradient.

5. The membrane according to claim 1, wherein the layer of fabric (N1, N1′, N2, N3; N61, N62, N63, N64, N65) is disposed on a substrate (S1, S2, S3).

6. The membrane according to claim 1, comprising two layers of fabric (N1, N1′, N2, N3) disposed on either side of a substrate (S1, S2, S3).

7. The membrane according to claim 4, wherein the substrate (S1, S2, S3) is less porous than the layer of fabric (N1, N1′, N2, N3).

8. The membrane according to claim 4, 5 or 6, wherein the substrate (S1, S2, S3) is chosen from among:
a poly(ε-caprolactone) (PCL) film,
a poly(dioxanone) (PDO) film,
a polyethylene terephthalate (PET) film,
a polypropylene (PP) film,
a non-woven fabric web, advantageously made of poly(ε-caprolactone) (PCL) and/or poly(dioxanone) (PDO), having a greater fibre density than that of the layer of fabric (N1, N1′, N2, N3).

9. A multilayer structure (M1; M2) comprising:
a ceramic substrate (S5), advantageously tricalcium phosphate, and preferably tricalcium β-phosphate (βTCP),
a membrane according to claim 1, advantageously made of poly(ε-caprolactone) (PCL), and
optionally, a cohesion interface (Ic), disposed between the ceramic substrate (S5) and the membrane.

10. A dressing intended to be applied onto a skin wound, the dressing comprising a substrate (S6) and at least one membrane according to claim 1.

11. A method of manufacturing a membrane, used in particular for regenerating the periodontal ligament, comprising at least one layer of fabric (N1, N1′, N2, N3; N4; N61, N62, N63, N64, N65) made of fibres (F) produced from at least one polymer, the method comprising the following steps:
a) obtaining polymer fibres (F) free of sizing,
b) depositing these polymer fibres (F) in a random or aligned manner, so as to form a non-woven layer of fabric (N1, N1′, N2, N3; N4; N61, N62, N63, N64, N65),
c) applying PolyNaSS (polystyrene sodium sulfonate) on the fibres of the non-woven layer of fabric (N1, N1′, N2, N3; N4; N61, N62, N63, N64, N65).

12. The method according to claim 11, wherein the polymer is selected from poly(ε-caprolactone) (PCL), poly(dioxanone) (PDO), a copolymer of poly(ε-caprolactone) (PCL) and poly (dioxanone) (PDO), polyethylene glycol terephthalate (PET), and polypropylene (PP).

13. The method according to claim 11, wherein a non-woven layer of fabric (N1, N1′, N2, N3) is deposited on one or both sides of a substrate (S1, S2, S3) selected from among:
a poly(ε-caprolactone) (PCL) film,
a poly(dioxanone) (PDO) film,
a polyethylene terephthalate (PET) film,
a polypropylene (PP) film,
a non-woven fabric web, advantageously made of poly(ε-caprolactone) (PCL) and/or poly(dioxanone) (PDO), having a greater fibre density than that of the layer of fabric (N1, N1′, N2, N3).
